(19) United States
(12) Patent Application Publication
(10) Pub. No.: US 2002/0164294 A1
(43) Pub. Date: Nov. 7, 2002

(54) Methods for producing a matte effect

(76) Inventors: Kalliope Christophides-Lordi, Clark, NJ (US); Maria Ulrich, Wayne, NJ (US)

Correspondence Address: FINNEGAN, HENDERSON, FARABOW, GARRETT &, DUNNER LLP, 1300 I STREET, NW, WASHINGTON, DC, 20005, US

(21) Appl. No.: 09/784,210

(22) Filed: Feb. 16, 2001

(51) Int. Cl. A61K 7/025

(52) U.S. Cl. 424/64

(57) ABSTRACT

A method for producing a matte effect on a substrate to which a cosmetic composition is applied, by applying to the substrate a cosmetic composition containing tricontanyl PVP in an amount sufficient to produce a matte effect on the substrate. The tricontanyl PVP may further provide the cosmetic composition with long-wearing and non-drying properties.

A subject of the present invention is a method for producing a matte effect on a substrate to which a cosmetic composition is applied. According to the present invention, the matte effect may be produced by applying a cosmetic composition containing tricontanyl PVP in an amount sufficient to produce a matte effect on the substrate. The cosmetic composition may be provided in the form of a stick and may be used for the care of and/or as make-up for the skin and the semi-mucous membranes, and in particular the lips.

By definition, a matte-effect product is a product which prevents the skin or lips from shining and/or which evens out the complexion. Care or make-up compositions for the skin or lips which have matte-effect properties are generally used to solve the problems of sheen caused by an excess of sebum and to improve the long-term staying power of make-up, which has a tendency to become visually degraded in the course of a day. These compositions can give the skin or lips a matte appearance resulting from the powerful reflection of light at the surface of the skin or lips. They can also be used to fade out defects or imperfections in the skin such as microreliefs, wrinkles, fine lines, pores or color variations. A matte effect is particularly sought by users with mixed or greasy skin, as well as for use in hot and humid climates.

Conventional matte-effect compositions generally contain powders which adsorb sebum and the excess oil in the composition not adsorbed by the skin. Among the matte-effect powders of natural or synthetic origin which may be mentioned in particular are fillers such as talc, starch, mica, silica, nylon powders, polyethylene powders, poly-beta-alanine powders and polymethyl (meth)-acrylate powders. However, fillers of this type have the drawback of giving the skin or lips an unnatural powdery appearance, which may even accentuate its defects. Also, these compositions generally cause long-term drying out of the skin or lips and are difficult to smooth on these substrates. Their matte effect is also short-lived. Further, there are difficulties associated with introducing such powders or fillers into compositions wherein the primary ingredient is a viscous oil (e.g., lanolin oil, which may be used to give comfort and shine to lipsticks) because the composition may become too thick and entrap air during molding.

There is thus a need for a matte-effect composition with a comfortable feel and light texture. Even more advantageous would be a matte effect composition whose properties persist on the skin or lips over time (i.e., long wearing), but which is non-drying as well.

The present inventors have discovered, surprisingly, that the incorporation of tricontanyl PVP in an amount sufficient to produce a matte effect into a composition, in particular a cosmetic composition, allows at least one of these problems to be solved, and also may result in a non-drying, long-wearing cosmetic composition. The non-drying aspect of the invention makes the composition comfortable to wear.

Accordingly, an embodiment of the present invention is drawn to a method for producing a matte effect on a substrate to which a cosmetic composition is applied, by applying to the substrate a cosmetic composition comprising tricontanyl PVP in an amount sufficient to produce a matte effect on the substrate. In another, related, embodiment, the amount of tricontanyl PVP used (i.e., the amount sufficient to produce a matte effect) not only produces a matte effect but further provides the cosmetic composition with long-wearing and non-drying properties.

Tricontanyl PVP is a polymer of vinylpyrrolidone and 1-triacontene having repeating units of the the following formula (I):

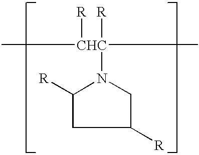

wherein R represents the triacontene moiety or hydrogen. Tricontanyl PVP (its CTFA name) is also known as 2-pyrrolidone, 1-ethenyl polymer with 1-triacontene. It is available commercially from ISP under the tradenames ANTARON WP-660 and GANEX WP-660. Tricontanyl PVP is known as a film former or viscosity increasing agent.

In the present invention, tricontanyl-PVP is used in an amount sufficient to produce a matte effect on the substrate to which it is applied. The substrate may be chosen from skin and semi-mucous membranes, such as the lips. In one embodiment, the tricontanyl-PVP is used in the present invention in an amount less than 3% by weight relative to the total weight of the composition. In another embodiment, the tricontanyl-PVP is present in an amount ranging from 0.1 to 2% by weight, such as 0.5% by weight.

The amount of tricontanyl-PVP sufficient to produce a matte effect may also result in a cosmetic composition with long-wearing and non-drying properties. “Non-drying” is defined herein to mean that the cosmetic composition, once applied to the substrate, retained a light, comfortable feel, without making the substrate, e.g., skin or lips, feel dry. “Long-wearing” is defined herein to mean that the cosmetic composition retained its color and comfortable feel for a significant amount of time, e.g., for at least 4 continuous hours.

The cosmetic compositions of the present invention may further comprise at least one wax and/or at least one oil. As used herein, “at least one” means one or more and thus includes individual components as well as mixtures/combinations. Non-limiting examples of waxes useful in cosmetic compositions include waxes of natural origin, such as beeswax, carnauba wax, candelilla wax, ouricury wax, Japan wax, cork fiber wax, sugar cane wax, and lanolin wax, hydrogenated oils such as hydrogenated jojoba oil, jojoba esters, waxes of synthetic origin, such as paraffin waxes, lignite wax, microcrystalline waxes, montan wax, ozokerites, polyethylene waxes derived from polymerization of ethylene, waxes obtained by Fischer-Tropsch synthesis, fatty acid esters and glycerides, and silicone waxes such as derivatives of poly(di)methylsiloxane. In one embodiment, the at least one wax may be present in the composition in an amount ranging from 1 % to 25% relative to the total weight of the cosmetic composition, and in another embodiment from 5% to 15%.

Non-limiting examples of oils useful in cosmetic compositions include polar oils and apolar oils including hydrocarbon-based liquid oils and oily liquids at room temperature. For example, the at least one polar oil useful in the invention may be chosen from:

hydrocarbon-based plant oils with a high content of triglycerides comprising fatty acid esters of glycerol in which the fatty acids may have varied chain lengths from C₄ to C₂₄, these chains possibly being chosen from linear and branched, and saturated and unsaturated chains; these oils can be chosen from, for example, wheat germ oil, corn oil, sunflower oil, karite butter, castor oil, sweet almond oil, macadamia oil, apricot oil, soybean oil, cotton oil, alfalfa oil, poppy oil, pumpkin oil, sesame oil, marrow oil, rapeseed oil, avocado oil, hazelnut oil, grape seed oil, blackcurrant seed oil, evening primrose oil, millet oil, barley oil, quinoa oil, olive oil, rye oil, safflower oil, candlenut oil, passion flower oil and musk rose oil; or alternatively caprylic/capric acid;

synthetic oils or esters of formula R₅COOR₆ in which R₅ is chosen from linear and branched fatty acid residues containing from 1 to 40 carbon atoms, e.g., from 1 to 12 carbon atoms, and R₆ is chosen from, for example, a hydrocarbon-based chain containing from 1 to 40 carbon atoms, e.g., from 1 to 12 carbon atoms, such as, for example, purcellin oil (cetostearyl octanoate), isononyl isononanoate, C₁₂-C₁₅ alkyl benzoates, isopropyl myristate, 2-ethylhexyl palmitate, isostearyl isostearate and alkyl or polyalkyl octanoates, decanoates or ricinoleates; hydroxylated esters such as isostearyl lactate and diisostearyl maleate; and pentaerythritol esters;

synthetic ethers containing from 10 to 40 carbon atoms;

C₈ to C₂₆ fatty alcohols such as oleyl alcohol; and

C₈ to C₂₆ fatty acids such as oleic acid, linolenic acid or linoleic acid.

The at least one apolar oil according to the invention may be chosen from, for example, silicone oils chosen from volatile and non-volatile, linear and cyclic polydimethylsiloxanes (PDMSs) that are liquid at room temperature; polydimethylsiloxanes comprising alkyl or alkoxy groups which are pendant and/or at the end of the silicone chain, the groups each containing from 2 to 24 carbon atoms; phenylsilicones such as phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates; hydrocarbons chosen from linear and branched, volatile and non-volatile hydrocarbons of synthetic and mineral origin, such as volatile liquid paraffins (such as isoparaffins and isododecane) or non-volatile liquid paraffins and derivatives thereof, liquid petrolatum, liquid lanolin, polydecenes, hydrogenated polyisobutene such as Parleam®, and squalane; and mixtures thereof. In one embodiment, the at least one oil may be present in the composition in an amount ranging from 1% to 75% relative to the total weight of the cosmetic composition, and in another embodiment from 10% to 50%.

In one embodiment, the compositions of the present invention may comprise hydrocarbon-based plant oils chosen from castor oil, a natural triglyceride which is lighter and less viscous than other oils and particularly useful in providing a light comfortable texture for the composition.

In another embodiment, the compositions of the invention may be lipstick compositions comprising at least one ester of formula R₅COOR₆, in which R₅ is chosen from linear and branched fatty acid residues containing from 1 to 12 carbon atoms, and R₆ is chosen from, for example, a hydrocarbon-based chain containing from 1 to 12 carbon atoms. As used herein, these esters may also be called “short-chain esters.” These short chain esters have a light powdery feel on the skin. Typically these lightweight short-chain esters are hard to incorporate in a lipstick composition because they tend to migrate to the surface of the stick, causing syneresis. However, if at least one bentone, such as quaternium 18 hectorite (available under the tradename Bentone 38), stearalkonium hectorite (available under the tradename Bentone 27), or mixtures thereof (available under the tradename category Bentone Gel from Rheox), is added to the lipstick formulation, a stabilizing “network” is formed within the composition, trapping the esters within it and preventing syneresis. In one embodiment, the at least one bentone may be present in the composition in an amount ranging from 0.1% to 3%, and in another embodiment from 0.5% to 2%.

Thus, in one embodiment, lipstick compositions of the invention comprise at least one hydrocarbon-based plant oil, such as castor oil, at least one short-chain ester, and at least one bentone.

The composition may also further comprise at least one suitable additive commonly used in cosmetic compositions and chosen from silica, e.g., fumed silica, which may contribute to the texture and comfort of a lipstick, fillers, pearlescent agents, coloring agents such as pigments, antioxidants, essential oils, preserving agents, fragrances, neutralizing agents, liposoluble polymers, and cosmetically active agents and dermatological active agents such as, for example, emollients, moisturizers, vitamins, essential fatty acids and sunscreens. The at least one additive is generally present in a concentration ranging from 0.01% to 35% by weight of the total weight of the composition.

In one embodiment, the composition of the present invention is in the form of a stick. For example, the composition may be in the form of a foundation, concealer, blush, or lipstick. In another embodiment, the composition of the invention may be anhydrous.

All numbers expressing quantities, reaction conditions, and so forth used herein are to be understood as being modified in all instances by the term “about.” Accordingly, unless indicated to the contrary, the numerical parameters set forth in the specification herein and in the attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The invention will be illustrated by, but is not intended to be limited to, the following examples.

### EXAMPLE 1

The following anhydrous lipstick composition was prepared:

| INGREDIENT | AMOUNT WT % |
|---|---|
| Oil Phase* | 65.53% |
| Vitamins (including antioxidants) | 0.50% |
| Waxes | 12.90% |
| Tricontanyl-PVP | 0.50% |
| Cholesterol | 0.10% |
| Silica Dimethyl Silylate | 2.00% |
| Pigments and Fillers | 18.24% |
| 2-oleamido-1,3-octadecanediol | 0.05% |
| Fragrance | 0.18% |
| Tricaprylin | 22.84% |
| Cetyl Ethylhexanoate | 22.92% |
| Cetyl Acetate (and) Acetylated Lanolin Alcohol | 3.05% |
| Isononyl Isononanoate | 22.84% |
| Ricinus communis (castor) seed oil | 26.96% |
| Stearalkonium hectorite | 1.07% |
| Propylene carbonate | 0.32% |

### EXAMPLE 2

Three lipstick compositions were formulated, generally following the ingredient list above. Specifically, composition A contained 0.5% tricontanyl-PVP, composition B contained no tricontanyl-PVP, and composition C contained 0.5% of the polymer polybutene. The lipsticks were applied to a laminated drawdown card and the gloss properties of the resulting film were measured with a gloss meter (BYK Gardener, micro-PRI-gloss, Model 4525). The results shown below are expressed in % reflectance. The lower the percentage, the more matte (less glossy) the film. Seven to nine readings were taken for each composition and an average was then taken.

| Composition A | Composition B | Composition C |
|---|---|---|
| (inventive) | (comparative) | (comparative) |
| 3.2 | 7.0 | 6.7 |
| 3.8 | 11.5 | 10.2 |
| 6.5 | 11.6 | 12.5 |
| 4.9 | 9.5 | 13.4 |
| 3.1 | 7.4 | 16.1 |
| 5.2 | 8.8 | 17.1 |
| 5.1 | 8.5 | 14.2 |
| — | 9.6 | — |
| — | 9.5 | — |
| AVERAGE: 4.54 | AVERAGE: 9.26 | AVERAGE: 12.89 |

As is clear from the table, the gloss was much lower for inventive composition A, which contained the 0.5% tricontanyl-PVP, indicating the presence of a matte effect for this lipstick.

What is claimed is:

1. A method for producing a matte effect on a substrate to which a cosmetic composition is applied, comprising applying to said substrate a cosmetic composition comprising tricontanyl PVP in an amount sufficient to produce a matte effect on said substrate.

2. A method according to claim 1, wherein said tricontanyl PVP is present in said cosmetic composition in an amount of less than 3% by weight, relative to the total weight of the composition.

3. A method according to claim 2, wherein said tricontanyl PVP is present in said cosmetic composition in an amount ranging from 0.1 to 2% by weight, relative to the total weight of the composition.

4. A method according to claim 3, wherein said tricontanyl PVP is present in said cosmetic composition in an amount of 0.5% by weight, relative to the total weight of the composition.

5. A method according to claim 1, wherein said substrate is chosen from skin and semi-mucous membranes.

6. A method according to claim 4, wherein said semi-mucous membranes are the lips.

7. A method according to claim 1, wherein said composition further comprises at least one wax.

8. A method according to claim 7, wherein said at least one wax is present in said composition in an amount ranging from 1% to 25% relative to the total weight of the composition.

9. A method according to claim 8, wherein said at least one wax is present in said composition in an amount ranging from 5% to 15% relative to the total weight of the composition.

10. A method according to claim 1, wherein said composition further comprises at least one oil.

11. A method according to claim 10, wherein said at least one oil is chosen from at least one polar oil and at least one apolar oil.

12. A method according to claim 11, wherein said at least one polar oil is chosen from hydrocarbon-based plant oils, synthetic oils, and esters of formula R₅COOR₆, wherein R₅ is chosen from linear and branched fatty acid residues containing from 1 to 12 carbon atoms and R₆ is chosen from a hydrocarbon-based chain containing from 1 to 12 carbon atoms.

13. A method according to claim 12, wherein said at least one polar oil is present in said composition in an amount ranging from 1% to 75% relative to the total weight of the cosmetic composition.

14. A method according to claim 13, wherein said at least one polar oil is present in said composition in an amount ranging from 10% to 50% relative to the total weight of the cosmetic composition

15. A method according to claim 1, wherein said cosmetic composition further comprises at least one bentone.

16. A method according to claim 15, wherein said bentone is present in an amount ranging from 0.1% to 3.0% relative to the total weight of the composition.

17. A method according to claim 16, wherein said bentone is present in an amount ranging from 0.5% to 2.0% relative to the total weight of the composition.

18. A method according to claim 1, wherein said cosmetic composition further comprises at least one hydrocarbon-based plant oil, at least one ester of formula R₅COOR₆, wherein R₅ is chosen from linear and branched fatty acid residues containing from 1 to 12 carbon atoms and R₆ is chosen from a hydrocarbon-based chain containing from 1 to 12 carbon atoms, and at least one bentone.

19. A method according to claim 1, wherein said cosmetic composition is in the form of a stick.

20. A method according to claim 1, wherein said cosmetic composition is in the form of a foundation, blush, concealer, or lipstick.

21. A method according to claim 1, wherein said cosmetic composition is anhydrous.

22. A method for producing a matte effect on a substrate to which a cosmetic composition is applied, comprising applying to said substrate a cosmetic composition comprising tricontanyl PVP in an amount sufficient to produce a matte effect on said substrate, and wherein said amount of tricontanyl PVP further provides said cosmetic composition with long-wearing and non-drying properties.

23. A method according to claim 22, wherein said tricontanyl PVP is present in said cosmetic composition in an amount of less than 3% by weight, relative to the total weight of the composition.

24. A method according to claim 23, wherein said tricontanyl PVP is present in said cosmetic composition in an amount ranging from 0.1 to 2% by weight, relative to the total weight of the composition.

25. A method according to claim 24, wherein said tricontanyl PVP is present in said cosmetic composition in an amount of 0.5% by weight, relative to the total weight of the composition.

26. A method according to claim 22, wherein said substrate is chosen from skin and semi-mucous membranes.

27. A method according to claim 26, wherein said semi-mucous membranes are the lips.

28. A method according to claim 22, wherein said composition further comprises at least one wax.

29. A method according to claim 28, wherein said at least one wax is present in said composition in an amount ranging from 1% to 25% relative to the total weight of the composition.

30. A method according to claim 29, wherein said at least one wax is present in said composition in an amount ranging from 5% to 15% relative to the total weight of the composition.

31. A method according to claim 22, wherein said composition further comprises at least one oil.

32. A method according to claim 31, wherein said at least one oil is chosen from at least one polar oil and at least one apolar oil.

33. A method according to claim 32, wherein said at least one polar oil is chosen from hydrocarbon-based plant oils, synthetic oils, and esters of formula R₅COOR₆, wherein R₅ is chosen from linear and branched fatty acid residues containing from 1 to 12 carbon atoms and R₆ is chosen from a hydrocarbon-based chain containing from 1 to 12 carbon atoms.

34. A method according to claim 33, wherein said at least one polar oil is present in said composition in an amount ranging from 1% to 75% relative to the total weight of the cosmetic composition.

35. A method according to claim 34, wherein said at least one polar oil is present in said composition in an amount ranging from 10% to 50% relative to the total weight of the cosmetic composition

36. A method according to claim 22, wherein said cosmetic composition further comprises at least one bentone.

37. A method according to claim 36, wherein said bentone is present in an amount ranging from 0.1% to 3.0% relative to the total weight of the composition.

38. A method according to claim 37, wherein said bentone is present in an amount ranging from 0.5% to 2.0% relative to the total weight of the composition.

39. A method according to claim 22, wherein said cosmetic composition further comprises at least one hydrocarbon-based plant oil, at least one ester of formula R₅COOR₆, wherein R₅ is chosen from linear and branched fatty acid residues containing from 1 to 12 carbon atoms and R₆ is chosen from a hydrocarbon-based chain containing from 1 to 12 carbon atoms, and at least one bentone.

40. A method according to claim 22, wherein said cosmetic composition is in the form of a stick.

41. A method according to claim 40, wherein said cosmetic composition is in the form of a foundation, blush, concealer, or lipstick.

42. A method according to claim 22, wherein said cosmetic composition is anhydrous.
